(19) United States
(12) Patent Application Publication
(10) Pub. No.: US 2010/0330141 A1
(43) Pub. Date: Dec. 30, 2010

(54) PRODUCT FOR ABSORPTION PURPOSES

(76) Inventors: Torkel WADSTRÖM, Lund (SE); Asa LJUNGH, Lund (SE); Marie-Christine HJERTÉN, Uppsala (SE); Nils-Gunnar LLBÄCK, Uppsala (SE); Stellan HJERTÉN, Uppsala (SE)

Correspondence Address: YOUNG & THOMPSON, 209 Madison Street, Suite 500, Alexandria, VA, 22314, US

(21) Appl. No.: 12/880,728

(22) Filed: Sep. 13, 2010

Related U.S. Application Data
(62) Division of application No. 10/559,464, filed on Mar. 9, 2006.
(60) Provisional application No. 60/477,960, filed on Jun. 13, 2003.

(51) Int. Cl. A01N 25/34; A61K 9/00; A01N 25/00; A01N 33/00; A01P 1/00

(52) U.S. Cl. 424/411; 424/486; 424/78.08; 424/445

(57) ABSTRACT

A product for absorption purposes comprises a water insoluble support matrix substituted with a hydrophobic entity and a positively charged entity. The hydrophobic entity comprises di-alkyl carbamoyl chloride (DACC) and the positively charged entity comprises polyethyleneimine (PEI). The product is capable of removing microorganisms, including viruses, from air and liquid. Methods for manufacturing the product and uses and applications of the product are also presented.

The present invention relates to a product for absorption purposes, preferably for the absorption of airborne and/or liquid borne microbes as well as viruses, and microbial antigens including allergens (which may be fungal), comprising an in water Insoluble support matrix which is connected to a hydrophobic entity which in turn is connected to a positively charged entity (other than said in water insoluble support matrix). As a support matrix an organic polymer or a combination of such e.g. polysaccharides such as cellulose etc, may be used. The present invention aims for achieving an improved absorbent which binds, preferably airborne and/or liquid borne, microorganisms such as bacteria as well as viruses, which also preferably may be airborne and/or liquid borne, and/or allergens.

### BACKGROUND

An increasing problem of airborne microbes and viruses (e.g. Influenza and SARS) and microbial antigens in airborne infections and associated diseases such as asthma has encouraged the development of a more effective method to remove these agents and antigens (i) from a highly contaminated environment, (ii) environments with conventional air filters, such as hospital operating theatres and hospital ward rooms for severely immuno-suppressed patients, and (iii) by personal face masks for hospital personnel. Such new equipment would be of great importance in modem hospital care with numerous patients highly sensitive for infections, e.g. in hematology, oncology and transplantation units.

The present Invention discloses, among other things, products, e.g. barriers/filters, to trap airborne and/or liquid borne bacteria, viruses and fungi, for which no efficient such barriers/filters exist, for protecting patients, hospital personnel and people in general during epidemics. It can also be applied on surgical equipment and in showers for immuno-suppressed patients.

There have been filters disclosed in U.S. Pat. No. 4,883,052, U.S. Pat. No. 5,817,584, U.S. Pat. No. 6,412,486, U.S. Pat. No. 6,119,691 and U.S. Pat. No. 4,985,280, but no one of the disclosed filters makes use of the fact that all microbes are negatively charged and that most pathogenic microbes and viruses express strong or moderate cell surface hydrophobicity and accordingly these filters provide less efficient absorption of said microbes and viruses. The very same principle may be used to trap airborne and/or liquid borne allergens. Accordingly, it would be useful with new more efficient absorbing materials using the combination of facts as set out above i.e. that all microbes and viruses are negatively charged and that most pathogenic microbes and viruses express strong or moderate cell surface hydrophobicity.

### SUMMARY OF THE INVENTION

These objects, e.g. solving the above problem with less efficient absorption as set out above, are achieved and further advantages are obtained with the absorbent according to the invention which in its most common embodiment is based upon a support matrix, which may consist of a polysaccharide, or other material as set out below, in the present description, to which different entities are connected.

The present invention thus solves the above problems by providing, according to a first aspect, a product for absorption purposes consisting of an in water insoluble support matrix wherein the support matrix is substituted with a hydrophobic entity which in turn is connected to a positively charged entity (other than said in water insoluble support matrix). According to a second aspect a method for the manufacture of a product according to the first aspect is provided, wherein a hydrophobic entity connected to a positively charged entity, is attached to a support matrix, preferably using an elimination reaction involving a good leaving group on the hydrophobic entity and a high pH. According to a third aspect of the present invention there is also provided a product obtainable by a method according to the second aspect. According to a fourth aspect of the present invention there is also provided use of a product according to the first aspect or third aspect for absorbing microorganisms, preferably airborne and/or liquid borne microorganisms, as well as viruses, preferably airborne and/or liquid borne viruses, and also allergens. According to a fifth aspect of the present invention there is also provided a face mask comprising a product according to the first aspect or third aspect. According to a sixth aspect of the present invention there is also provided a wound dressing (compress) comprising a product according to the first aspect or third aspect. According to a seventh aspect of the present invention there is also provided a drape for use during a surgical intervention comprising a product according to the first aspect or third aspect. According to an eighth aspect of the present invention there is also provided a filter comprising a product according to the first aspect or third aspect. The filter is then both hydrophobic and positively charged. According to a ninth aspect of the present invention there is also provided a “tea bag”, preferably for obtaining potable water by dipping said tea bag into non-potable water, comprising a product according to the first aspect or third aspect. According to a tenth aspect of the present invention there is also provided a nasal spray comprising a product according to the first aspect or third aspect for capturing microorganisms, preferably airborne and/or liquid borne microorganisms, as well as viruses, preferably airborne and/or liquid borne viruses in the nasal cavity. According to a eleventh aspect of the present invention there is also provided an ointment comprising a product according to the first aspect or third aspect for capturing microorganisms, preferably airborne and/or liquid borne microorganisms, as well as viruses, preferably airborne and/or liquid borne viruses on the skin of animals or humans. Said ointment may in addition to said product according to the first aspect or third aspect comprise other components for use in ointments and said additional components for use in ointments are known for a person skilled in the art. According to a twelfth aspect of the present invention there is also provided medical devices, e.g. catheters for drainage and rinsing of the urinary and genital tracts, comprising a product according to the first aspect or third aspect. Preferably said device is a catheter which may be used in the urinary, genital or respiratory tracts. According to a fourteenth aspect of the present invention there is also provided filter arrangement comprising two filters according to the eighth aspect wherein said filters are having in between them one or more products according to the first or third aspect in particulate form, thus enabling a larger surface area for absorption. According to a fifteenth aspect of the present invention there is also provided a food wrapping and an active food packaging material comprising a product according to the first aspect or third aspect; said product may preferably comprise in its support matrix a polysaccharide in its native state, positively charged.

### DETAILED DESCRIPTION OF THE INVENTION

It is intended throughout the present description that the expression “support matrix” embraces any matrix which is built up of an in water insoluble polymer material. Examples thereof are agarose particles, agar particles and polygalactanes (comprising polygalactose units), agarose or derivatives thereof, laminarine, cellulose (e.g. cotton) or derivatives thereof, cross-linked dextrane or derivatives thereof, and starch or derivatives thereof. Preferably materials commonly used in filter or face masks are used as support matrix. An example thereof is cellulose, which is the most preferred. A polysaccharide such as agarose and cellulose may be regarded as thread-shaped molecules consisting of monomer units containing several hydroxyl groups and internal and external ether bonds (acetal bonds), which taken together give the polysaccharide affinity to water (it is said to be hydrophilic). Such polymers form in water swellable gels with hydroxyls as targets for substitution. It is also possible to use a polymeric backbone (preferably comprising polyethylene) covered with cellulose or similar material as a support matrix and this is especially preferred when using the absorption product according to the first aspect of the invention in face mask filters (the use of a backbone makes it easier to breath in said face mask) or in wound dressings. The support matrix may further be present in particulate form allowing the application of the product for absorption purposes according to the first or third aspect of the present invention by means of a nasal spray or an ointment.

According to a further embodiment of the first aspect of the present invention there Is provided a product wherein the hydrophobic entity is a saturated or unsaturated hydrocarbon chain with a chain length of from C₅ to C₂₅ or an aromatic group i.e. an alkyl or alkylene with a chain length of from C5 to C₂₈, preferably with a chain length of from C₈ to C₁₈, most preferred a saturated or unsaturated hydrocarbon chain with a chain length of from C₁₂ to C₁₈. The chain length may e.g. be C₁₅. The hydrocarbon chain may be included in a compound such as QUAB 342 (see below) or in QUAB 360 or QUAB 426 (which also comprise a positively charged group).

According to a further embodiment of the first aspect of the present invention, there is provided a product wherein the positively charged entity is a positively charged group, preferably an amino group or ammonium group. The ammonium group may be contained in a compound such as QUAB 342 (3-chloro-2-hydroxypropyl-climethyl-dodecyl-ammonium chloride) or in QUAB 360 and QUAB 426 (QUAB is a trade name of Degussa for solutions of the active substance 3-chloro-2-hydroxypropyl-dimethyl-alkyl-ammonium chloride. In said compound a methyl group within the quatemary ammonium group is substituted by a long-chain alkyl group (alkyl=dodecyl-, cocoalkyl- or stearyl-)). These compounds may be in the form of chlorohydrins. Instead of Cl- there may be a CCO-group instead (i.e. an epoxide group).

According to a further embodiment of the first aspect of the present invention there is provided a product wherein the support matrix comprises a polysaccharide, polygalactane, agar, agarose, laminarine, cellulose, crosslinked dextran, starch or a derivative thereof; or a mixture of two or more of said compounds; preferably said support matrix comprises cellulose.

According to a further embodiment of the first aspect of the present invention there is provided a product wherein the support matrix comprises a backbone covered with cellulose, preferably said backbone comprises a plastic material, most preferred polyethylene. The backbone may consist essentially of polyethylene only. Said product is especially useful in face mask filters (the use of a backbone makes it easier to breath in said face mask) or in wound dressings.

The hydrogel product according to the present invention may be manufactured as set out below (here cellulose is the support matrix):

| | |
|---|---|
| 1) Cellulose-OH + Cl—C₁₈H₃₆—NH₂ | (At a relatively high pH) |
| 2) Elimination of Cl⁻ | |
| 3) Cellulose-O—C₁₈H₃₈—NH₂ | (A product according to |
| | the first aspect of the invention) |

In the fourth aspect of the present invention, i.e. use of a product according to the first aspect or third aspect for absorbing microorganisms, preferably airborne and/or liquid borne microorganisms, as well as viruses, preferably airborne (in particular influenza viruses, SARS-virus) and/or liquid borne viruses, and also allergens, the airborne and/or liquid borne microorganisms may be wound pathogens such as Staphylococcus aureus, Group A beta-haemolytic streptococci, urinary catheter-related pathogens such as Escherichia coli, eczema-related pathogens such as Candida albicans and various bacteria, and bum pathogens such as Pseudomonas aeruginosa. Moreover, “new” pathogens prevalent particularly in the hospital setting are of interest, including methicillin-resistant Staphylococcus aureus (MRSA), vancomycin-resistant enterococci (VRE), Acinetobacter spp., multiresistant gramnegative intestinal bacteria, Stenotrophomonas maltophilia etc.

These microorganisms express high surface hydrophobicity and have been shown to bind to hydrophobic wound dressings.

According to a first variant of the first aspect of the present invention, which also solves the above problems, there is also provided a product for absorption purposes comprising a first support matrix connected to a hydrophobic entity and a second support matrix connected to a positively charged entity. Said product may be manufactured whereby a hydrophobic entity is connected to a first support matrix and a positively charged entity, is connected to a second support matrix, and said both support matrices are mixed. In said method the hydrophobic entity may be connected by using DACC (di-alkyl-carbamoyl-chloride), which is preferred, and the positively charged entity may be connected by using polyethyleneimine, which also is preferred. There is also provided a product obtainable by said method. Said products may be used in the same way and in the same applications as set out for the product according to the first aspect, as set out above. A hydrophobic filter may e.g. be combined with a positively charged filter to make it more effective.

According to a second variant of the first aspect of the present invention, which also solves the above problems, there is also provided a product for absorption purposes comprising a support matrix wherein the support matrix is substituted with a hydrophobic entity and a positively charged entity. Said product may be manufactured whereby a hydrophobic entity and a positively charged entity, is connected to a support matrix. In said method the hydrophobic entity may be connected by using DACC, which is preferred, and the positively charged entity may be connected by using polyethyleneimine, which also is preferred. It is also plausible that one of said entities is connected to said support matrix, thereby forming a semimanufactured article, and then the other entity is connected to said article whereby forming the end product. There is also provided a product obtainable by said method. Said products may be used in the same way and in the same applications as set out for the product according to the first aspect, as set out above.

It should be noted and recalled that all microbes as well as viruses are negatively charged and most pathogenic microbes as well as viruses express strong or moderate cell surface hydrophobicity. The same principle may be used to trap airborne and/or liquid borne allergens. A further advantage with the present invention is the possibility of capturing liquid borne microorganisms such as bacteria as well as viruses, which also preferably may be liquid borne, efficiently even if there is a high concentration of salts in the liquid where the microorganisms such as bacteria as well as viruses reside. Thus said liquid may have a varying content of salts ranging from virtually zero (water) to liquids comprising high concentration of salts. Said liquid may further be a buffer.

Depending on a great variety of applications for this new air-capture (for water applications, see below) different filters can be produced with varying filtering and absorbing capacities per gram material. The product of the first aspect may further be optimized for its binding capacity, e.g. when used in face masks humified by the normal breath. Similar products of the first aspect of the invention may further be used for water to capture water borne microbes, as well as viruses, and microbial and/or viral lysate products. Similar products of the first aspect of the invention may further be used food wrapping and active food packaging material for binding and absorbing contaminating bacteria in food, including microbial toxins and bacterial lysate products. Products according to the first or third aspects may also be used for absorbing bacteria and/or viruses and/or fungi in open wounds. These wound may be present on animals and on humans.

Preferred features of each aspect of the invention are as for each of the other aspects mutatis mutandis. The prior art documents mentioned herein are incorporated to the fullest extent permitted by law. The invention is further described in the following examples, which also refers to figures, which do not limit the scope of the invention in any way. Embodiments of the present invention are thus described in more detail with the aid of examples of embodiments (together with figures), the only purpose of which is to Illustrate the invention and are in no way intended to limit its extent.

### FIGURES

FIG. 1 shows density of Staphylococcus aureus in washing solution (H₂O) after use for washing a QUAB 342-non-treated control filter previously dipped in a solution containing 5×10⁷/ml of Staphylococcus aureus.

FIG. 2 shows density of Staphylococcus aureus in washing solution (H₂O) after use for washing a QUAB 342-treated filter previously dipped in a solution containing 5×10⁷/ml of Staphylococcus aureus.

FIG. 3 shows density of Escherichia coil in washing solution (H₂O) after use for washing a QUAB 342-non-treated control filter previously dipped in a solution containing 5×10⁷/ml of Escherichia coll.

FIG. 4 shows density of Escherichia coli in washing solution (H₂O) after use for washing a QUAB 342-treated filter previously dipped in a solution containing 5×10⁷/ml of Escherichia coli.

### EXAMPLES

### Example 1

We have hydrophobized various cellulose and similar fibers, conventionally used in filters, face masks as well as laboratory filters (Hepa filter, Camfil, Trosa, Bolinder Munktell). This has been done by coupling alkyl chains and aromatic groups by known methods.

### Example 2

In order to optimize binding of “new” and “old” pathogens such as methicillin resistant staphylococci (MRSA), vancomycin resistant enterococci (VRE), Acinetobacter sp, Stenotrophomonas maltophilla, prevalent in hospital settings, we modified the surface of hydrophobic filter polymer materials to make them also positively charged. Filter materials were analyzed for binding capacity of standard microbial aerosols obtained by conventional nebulizer for asthmatic medicines (Draco). Candida sp as well as Candida surface proteins (hydrophobins) were efficiently absorbed.

### Examples 3 and 4

In absorption studies of bacteria and viruses, cellulose filters treated in various ways were used. 1) The covalent binding of DACC (di-alkyl-carbamoyl-chloride) was used for studies on hydrophobic binding. 2) Electrostatic binding of polyethyleneimine in aqueous solution was used to obtain positively charged groups on cellulose. 3) Treatment of filters with QUAB 342, a compound which has both hydrophobic and positively charged groups and reactive CI atoms. QUAB 342=3-chloro-2-hydroxypropyl-dimethyl-dodecyl-ammonium chloride

Different regimens of washing of filters were used, i.e. de-ionized water or 0.9% NaCl. This procedure was used to study the impact of charge on the strength of the binding.

### Example 3

### Bacterial Adsorption

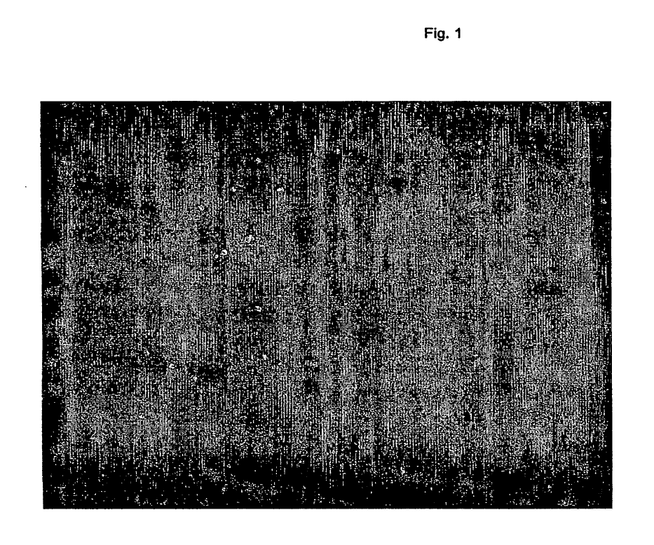

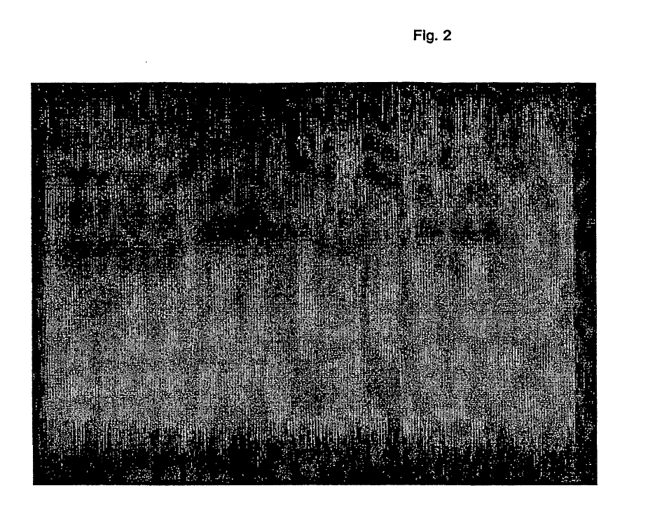

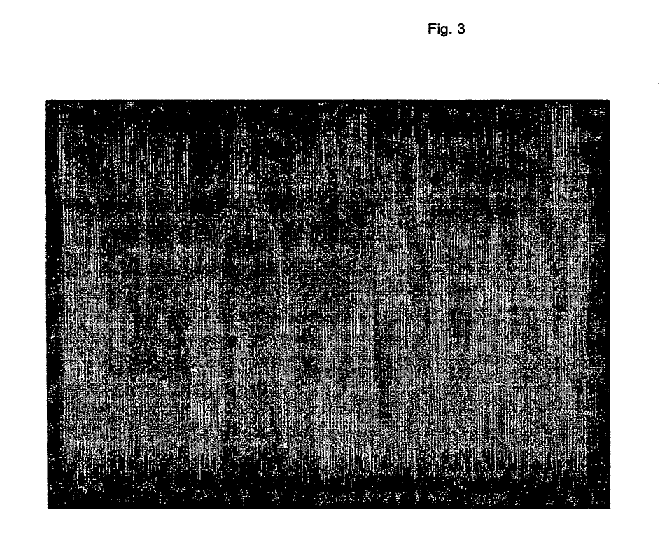

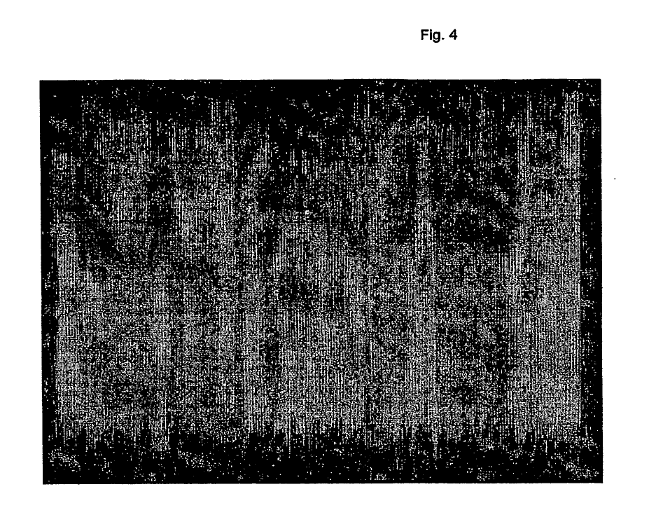

Pieces of non-treated and treated (QUAB 342) cellulose filters with a surface area of about 0.5 cm² were dipped during 1 min in solutions of different bacteria (5×10⁷/ml), i.e., Escherichia coli and Staphylococcus aureus. After dipping, the filters were washed during 1 min in 2 ml de-ionized water. Bacteria in the washing solution were stained with acridine-orange and quantitatively evaluated using fluorescence microscopy. Results showed that both bacteria were efficiently bound by the treated filters when compared to non-treated filters (see FIGS. 1-4).

### Example 4

### Adsorption of Virus to Cellulose Filters Treated in Different Ways

A. Pieces of non-treated and treated (DACC) cellulose filters with a surface area of about 0.5 cm² were dipped during 1 min in a solution of [³⁵S]-methionine/cysteine labelled coronavirus. Corona virus labeled with [³⁵S]-methionine/cysteine was accordingly used to test its binding (adsorption) to non-treated and treated cellulose filters. After dipping, the filters were washed twice during 1 min, either in de-ionized water or in 0.9% NaCl. After dipping and washing, the non-treated filters retained 4.3% (H₂O wash) and 4.6% (NaCl wash) of virus, whereas DACC-treated filters retained 12.9% (H₂O wash) and 21.4% (NaCl wash). The radioactivity of the non-treated and treated filters was 160 and 496 cpm., respectively during one wash regimen. DACC was added as dissolved in a 7.4.% aqueous solution of polyethyleneimine.

A cellulose filter (3 cm×5 cm) was also treated for one hour with DACC dissolved in a 14% aqueous solution of polyethyleneimine. The filter was then washed for 5 minutes with 50 ml water for each washing 4 times and was allowed to dry over night. At the same time as DACC was attached covalently polyethyleneimine was attached electrostatically to carboxylic groups in the cellulose filter.

B. Pieces of non-treated and treated (polyethyleneimine) filters were dipped and washed as in experiment A. After dipping and washing, the non-treated filters retained 11.5% (H₂O wash) and 6.7% (NaCl wash) of virus, whereas polyethyleneimine-treated filters retained 26% (H₂O wash) and 12.8% (NaCl wash). Thus, the experiment in A was repeated with the difference that the filters were not treated with DACC but with polyethyleneimine—water (1:1). The filters were dipped into a [³⁵S] labeled Corona virus solution and were washed 5 times with a physiological sodium chloride solution or water. The radioactivity of the non-treated and treated filters was 532 and 3211 cpm., respectively during one wash regimen.

C. Pieces of non-treated and treated (QUAB 342) cellulose filters were dipped and washed as in experiments A and B. After dipping and washing, the non-treated filters retained 36% (H₂O wash), whereas QUAB 342-treated filters retained 80% (H₂O wash) of the labelled virus. The experiment in A was thus repeated with the difference that the filters were not treated with DACC but QUAB 342 (a compound which has both a hydrophobic and a positively charged group and a reactive Cl atom) for 30 hours and they were then washed for 5 minutes 4 times with 50 ml of water and dried over night. The filters were then as above dipped into a [³⁵S]-methionine/cysteine labeled Corona virus solution. The radioactivity was 2202 cpm.

In addition, a blocking experiment was performed to show the specificity of the binding of virus. Non-treated and treated filters were pre-dipped in non-labelled virus solution prior to dipping in the labelled virus solution. After dipping in labelled virus solution, the filters were washed as previously. After this blocking experiment, the non-treated filter retained 24%, whereas the QUAB 342-treated filter retained 57% of the labelled virus. In conclusion, a clear-cut blocking effect by the non-labelled virus was shown, verifying the specificity of the binding.

Further similar experiments, involving virus, have been performed which gave similar results as the ones given above.

Various embodiments of the present invention have been described above but a person skilled in the art realizes further minor alterations, which would fall into the scope of the present invention. The breadth and scope of the present invention should not be limited by any of the above-described exemplary embodiments, but should be defined only in accordance with the following claims and their equivalents. For example, any of the above-noted products and/or methods can be combined with known therapies for treating microorganisms and/or viruses or compositions/products. Also any of the above-noted products and/or methods can be utilized in other areas than that of microorganisms and allergens for the removal of undesired particles and molecules. Other aspects, advantages and modifications within the scope of the invention will be apparent to those skilled in the art to which the invention pertains.

1. A product for absorption of microorganisms,
the product comprising a water insoluble support matrix substituted with:
a hydrophobic entity comprising di-alkyl carbamoyl chloride (DACC); and
a positively charged entity comprising polyethyleneimine (PEI).

2. The product according to claim 1, wherein the support matrix comprises polysaccharide, polygalactane, agar, agarose, laminarine, cellulose, crosslinked dextran, starch or a derivative thereof and mixtures thereof.

3. The product according to claim 1, wherein the DACC is covalently attached to the support matrix and the PEI is electrostatically attached to the support matrix.

4. A wound dressing comprising the product according to claim 1.

5. A face mask comprising the product according to claim 1.

6. A surgical drape comprising the product according to claim 1.

7. A tea bag comprising the product according to claim 1.

8. A nasal spray comprising the product according to claim 1.

9. An ointment comprising the product according to claim 1.

10. A medical device comprising the product according to claim 1.

11. A catheter comprising the product according to claim 1.

12. A food wrapping or food packaging material comprising the product according to claim 1.

13. A filter comprising the product according to claim 1.

14. A filter arrangement comprising:
two filters, each of the two filters comprising the product according to claim 1, and
said product in particulate form arranged in between the two filters.

15. A method of absorbing a microorganism from air or liquid comprising contacting the air or liquid with the product according to claim 1.

16. The method of claim 15, wherein the microorganism is a virus present in the air or liquid.

17. The method of claim 15, wherein the microorganism is an allergen present in the air or liquid.

18. The method of claim 15, wherein the microorganism is a bacteria present in the air or liquid.
